(19) United States
(12) Patent Application Publication
(10) Pub. No.: US 2017/0360555 A1
(43) Pub. Date: Dec. 21, 2017

(54) BREAST IMPLANTS

(71) Applicant: IMPLITE LTD., Petah Tikva (IL)

(72) Inventor: Ami GLICKSMAN, Petach Tikva (IL)

(21) Appl. No.: 15/533,599

(22) PCT Filed: Dec. 6, 2015
(86) PCT No.: PCT/IL2015/051183

§ 371 Date: Jun. 6, 2017

Related U.S. Application Data
(60) Provisional application No. 62/088,602, filed on Dec. 7, 2014.

(51) Int. Cl. A61F 2/12; A61F 2/00

(52) U.S. Cl. CPC A61F 2/12; A61F 2250/0004; A61F 2/0059; A61F 2230/0093

(57) ABSTRACT

There is provided herein a breast implant comprising: a base having a first diameter, the base is configured to rest against a subject's chest wall when implanted; a dome having a second diameter, the dome is configured to be positioned within breast parenchyma underneath a nipple-areola complex when implanted; and an elongated projecting structure extending between the base and the dome, wherein the implant is configured to be inserted into a subject's breast as an internal supporting skeleton and to affect the projection of breast.

### FIELD OF THE INVENTION

The present invention relates to breast implants, suitable inter alia for correcting breast sagging.

### BACKGROUND

Breast implants have been in use since the early 1960's. Since the first generations, all implants are generally made of a silicone shell filled with silicone gel, saline, soybean oil or other filler materials which are non-compressible in nature. When such implants are introduced during breast surgery into a sub-mammary or sub-pectoral pocket, both projection and volume increase of the breast are obtained. Thus, projection and augmentation are two inseparable effects of current breast implants in breast surgery.

Women's breasts differ in their volume, tissue density, consistency, and amount of Cooper's ligaments that pack and hold the breast tissue together and are responsible for keeping the breast from sagging. In terms of projection, breasts may be voluminous and erect or voluminous and sagging. Typically, later in life, when skin and tissue elasticity are reduced, breast sagging becomes more prominent. Nowadays, breast reshaping procedures utilize breast implants and various surgical techniques for skin envelope reduction and re-draping the remaining breast tissue, with or without a breast implant. As noted above, when breast implants are used, both projection and augmentation of the breast are obtained. However, breast augmentation is not always desired in breast reshaping procedures.

There is still a need in the art for breast implants and surgical procedures that enable correction of breast sagging separately from breast augmentation, to improve the results and efficiency of breast reshaping procedures.

The foregoing examples of the related art and limitations related therewith are intended to be illustrative and not exclusive. Other limitations of the related art will become apparent to those of skill in the art upon a reading of the specification and a study of the figures.

### SUMMARY OF INVENTION

The present invention provides, according to some aspects, breast implants that enable correction of breast sagging (known as breast ptosis) separately from breast augmentation. The breast implants, according to embodiments of the present invention, advantageously enable achieving breast projection without concomitant breast augmentation. The implant, according to some embodiments, acts as a resilient internal skeleton in the breast parenchyma, thus changing breast shape and maintaining it in a new contour.

According to one aspect, there is provided a breast implant (e.g., a three dimensional breast implant) comprising: a base having a first diameter, the base is configured to rest against a subject's chest wall when implanted; a dome having a second diameter, the dome is configured to be positioned within breast parenchyma underneath the nipple-areola complex when implanted; and an elongated projecting structure extending between the base and the dome, wherein the implant is configured to be inserted into a subject's breast as an internal supporting skeleton and to affect the projection of breast.

According to some embodiments, the elongated projecting structure may include a plurality of resilient elongated elements, one or more of the plurality of resilient elongated elements extend between the dome and the base. The elongated projecting structure may include one or more pillars. The elongated projecting structure may include at least one spring. The elongated projecting structure may include differently shaped projecting structures having different mechanical properties.

According to some embodiments, the implant or parts thereof may be formed of or comprise one or more resilient materials allowing natural movement of the breast. According to some embodiments, the elongated projecting structure may be formed of or comprise a resilient material.

According to some embodiments, the first diameter is larger than the second diameter. According to some embodiments, the elongated projecting structure and the dome may be integrally formed. According to some embodiments, at least one of the base, the projecting structure and the dome may be formed as a separate component, which is configured to be assembled with remaining component(s) to form the implant.

According to some embodiments, the implant may at least partially be made of human biocompatible materials. According to some embodiments, the implant may at least partially be coated with human biocompatible materials.

According to some embodiments, the dome may include one or more radial ribs.

According to some embodiments, the implant is configured to be assembled during surgery. According to some embodiments, the implant is variably configured to define projection of the breast during surgery. According to some embodiments, the implant is configured for altering the projection of the breast post-surgery.

According to some embodiments, an angle between the dome and the connecting projecting structure can be any angle. According to some embodiments, an angle between the base and the projecting structure can be any angle. According to some embodiments, these angels may variably be changed according to variable gravitational accelerations.

According to some embodiments, an angle between the dome and the projecting structure is changeable in response to applying external pressure by surrounding breast tissue.

According to some embodiments, an angle between the base and the projecting structure is changeable in response to applying external pressure by surrounding breast tissue.

According to some embodiments, the elongated projecting structure and the base are manufactured from materials having different mechanical and/or chemical properties.

According to some embodiments, the implant may further include a textured surface. According to some embodiments, the dome, the base, and the projecting structure include a body and an outer surface; the body and the outer surface are made from different materials.

According to some embodiments, the elongated projecting structure may be telescopic and may be configured to transiently change a distance between the dome and the base in response to pressure applied to the breast.

According to some embodiments, the implant includes a plurality of projecting structures, such that after implantation breast parenchyma is filling the space between at least two of the plurality of projecting structures. According to some embodiments, the elongated projecting structure may be changeable in response to pressure applied to it. According to some embodiments, the dome and the base may have different outer surface layer tactile feedback. According to some embodiments, the base may be substantially flat. According to some embodiments, the base may be substantially flat with its edges being curved downwardly. According to some embodiments, the base may be convex, having a smaller degree of convexity compared to the dome. According to some embodiments, the elongated projecting structure may be extending vertically between the base and the dome.

According to some embodiments, the implant may further include a torus structure configured to surround the projecting structure for further facilitating breast projection and for providing breast augmentation. According to some embodiments, the torus structure may be filled or fillable with a biocompatible material. The torus structure may be filled or fillable with a filler having specific gravity lower than 0.5 gr/cc. The torus structure may be filled or fillable with a filler having specific gravity lower than 0.4 gr/cc. The torus structure may be filled or fillable with a filler having specific gravity lower than 0.3 gr/cc.

According to some embodiments, the implant is a secondary implant for replacing a previous breast implant following extraction thereof, configured for placing inside a capsule formed in the breast around the previous breast implant.

According to another aspect, there is provided a breast implant kit comprising:
(i) an internal skeleton comprising: a base having a first diameter, the base is configured to rest against a subject's chest wall when implanted; a dome having a second diameter, the dome is configured to be positioned within breast parenchyma underneath the nipple-areola complex when implanted; and an elongated projecting structure extending between the base and the dome; and
(ii) a torus structure configured to surround the projecting structure and at least partially fill a gap between the base and the dome.

According to some embodiments, the kit further includes a biocompatible material for filling the torus structure.

According to yet another aspect, there is provided a method of affecting the projection of a breast, the method comprising: (i) inserting into the breast an internal skeleton comprising: a base having a first diameter, the base is configured to rest against a subject's chest wall when implanted; a dome having a second diameter, the dome is configured to be positioned within breast parenchyma underneath the nipple-areola complex when implanted; and an elongated projecting structure extending between the base and the dome; and (ii) adjusting breast projection.

According to some embodiments, the method further includes assembling the base, the projecting structure and the dome. According to some embodiments, the assembling is performed during surgery. According to some embodiments, the method further includes inserting a torus structure surrounding the projecting structure for further adjusting breast projection and for providing breast augmentation. According to some embodiments, the method further includes filling the torus structure with a filler.

More details and features of the current invention and its embodiments may be found in the description and the attached drawings.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting.

### BRIEF DESCRIPTION OF THE FIGURES

Exemplary embodiments are illustrated in referenced figures. Dimensions of components and features shown in the figures are generally chosen for convenience and clarity of presentation and are not necessarily shown to scale. It is intended that the embodiments and figures disclosed herein are to be considered illustrative rather than restrictive. The figures are listed below:

FIGS. 1A-R are illustrations of various views of different embodiments of a breast implant according to the present invention. FIG. 1A and FIG. 1B are respectively a sectional side view and a pictorial top view of an implant according to one embodiment of the present invention. FIG. 1C and FIG. 1D are sectional side views of other embodiments, relating to design and composition of various parts of the breast implant. FIG. 1E and FIG. 1F are sectional side views of additional embodiments, providing solutions for various breast shapes and weights. FIG. 1G and FIG. 1H are respectively a sectional side view and a pictorial top view of an implant according to another embodiment, allowing in-growth of tissue into various parts of the implant. FIG. 1I and FIG. 1J are similarly a sectional side view and a pictorial top view of an implant allowing tissue in-growth, further showing scar tissue formed therein. FIG. 1K and FIG. 1L are respectively a sectional side view and a pictorial top view of an implant according to an additional embodiment, comprising a spring. FIG. 1M and FIG. 1N are respectively a sectional side view and a pictorial top view of an implant according to an additional embodiment, comprising a curved projecting structure. FIG. 1O and FIG. 1P are respectively a sectional side view and a pictorial top view of an implant according to an additional embodiment, comprising a projecting structure composed of a plurality of resilient elongated elements. FIG. 1Q and FIG. 1R are respectively a sectional side view and a pictorial perspective view of an implant according to an additional embodiment, comprising a projecting structure composed of a plurality of sub-structures of different designs and properties.

FIGS. 2A-J are illustrations of lateral cross-sections of other embodiments of a breast implant according to the present invention comprising separate parts configured to assemble to one another. FIG. 2A and FIG. 2B illustrate a three-part implant design and assembly. FIG. 2C and FIG. 2D illustrate a three-part implant design and assembly, having a variable projection option. FIG. 2E and FIG. 2F illustrate an embodiment addressing breast tactility and consistency. FIGS. 2G-2J illustrate two embodiments of a collapsible projecting structure.

FIGS. 3A-D are illustrations of lateral cross-sections of yet other embodiments of a breast implant comprising separate parts configured to assemble to one another. FIG. 3A and FIG. 3B illustrate a three-part implant design and assembly. FIG. 3C and FIG. 3D illustrate another embodiment of a three-part implant design and assembly.

FIG. 4 is a lateral cross section illustration of a breast implant according to another embodiment of the present invention, comprising added layers for improved tactility of the dome part and the base part of the implant.

FIG. 5 is a schematic lateral section of a human breast in need of correction of breast sagging.

FIG. 6 is a schematic lateral section of a human breast with a projecting implant implanted therein, according to some embodiments.

FIG. 7 is a schematic lateral section of a human breast with a projecting implant implanted therein, with the addition of a torus augmenting structure surrounding the central projecting structure of the projecting implant, the implant is implanted for primary augmentation procedure, according to some embodiments.

FIG. 8 is a schematic lateral section of a human breast with a projecting implant implanted therein, with the addition of a torus augmenting structure surrounding the central projecting structure of the projecting implant, the implant is implanted as a secondary implant after extraction of a previous breast implant, according to some embodiments.

### DETAILED DESCRIPTION OF THE INVENTION

While a number of exemplary aspects and embodiments have been discussed above, those of skill in the art will recognize certain modifications, permutations, additions and sub-combinations thereof. It is therefore intended that the following appended claims and claims hereafter introduced be interpreted to include all such modifications, permutations, additions and sub-combinations as are within their true spirit and scope.

The following embodiments and aspects thereof are described and illustrated in conjunction with systems, tools and methods which are meant to be exemplary and illustrative, not limiting in scope.

In the description and claims of the application, each of the words “comprise” “include” and “have”, and forms thereof, are not necessarily limited to members in a list with which the words may be associated.

As used herein, the term “about”, when referring to a measurable value, is meant to encompass variations of +/−10%, more preferably +/−5%, even more preferably +/−1%, and still more preferably +/−0.1% from the specified value.

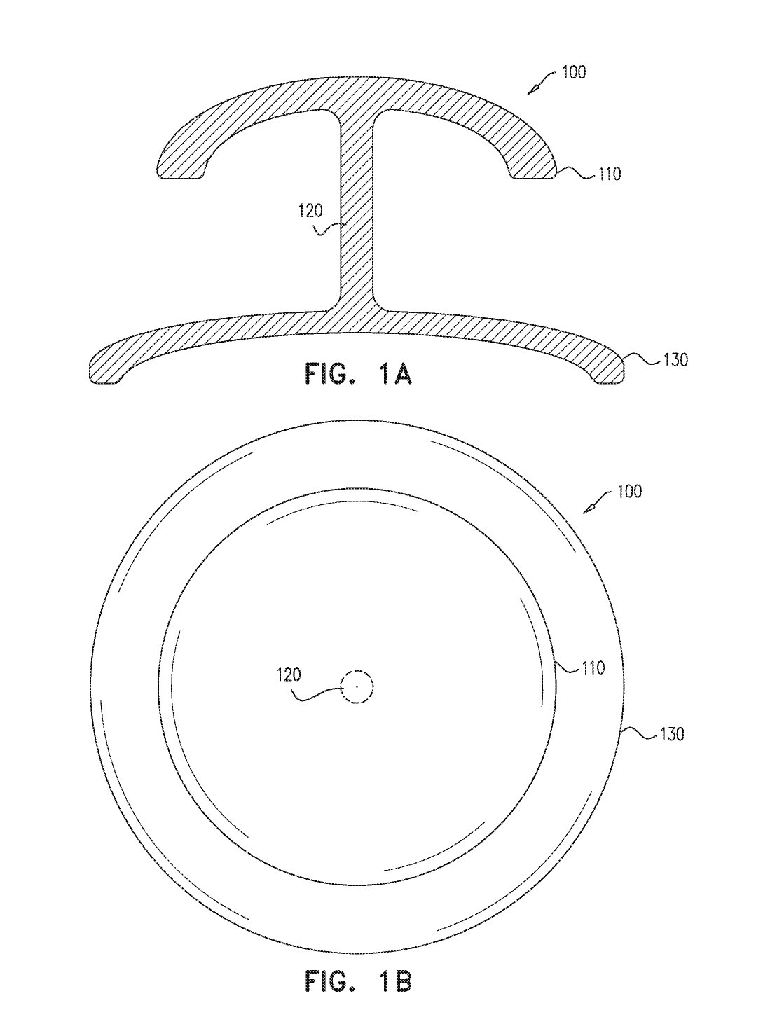

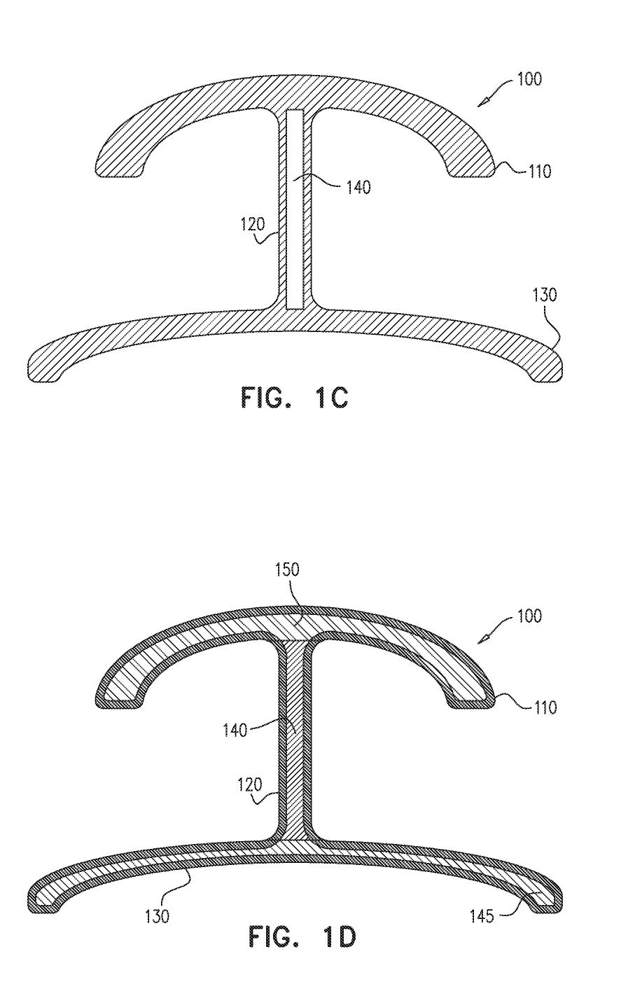

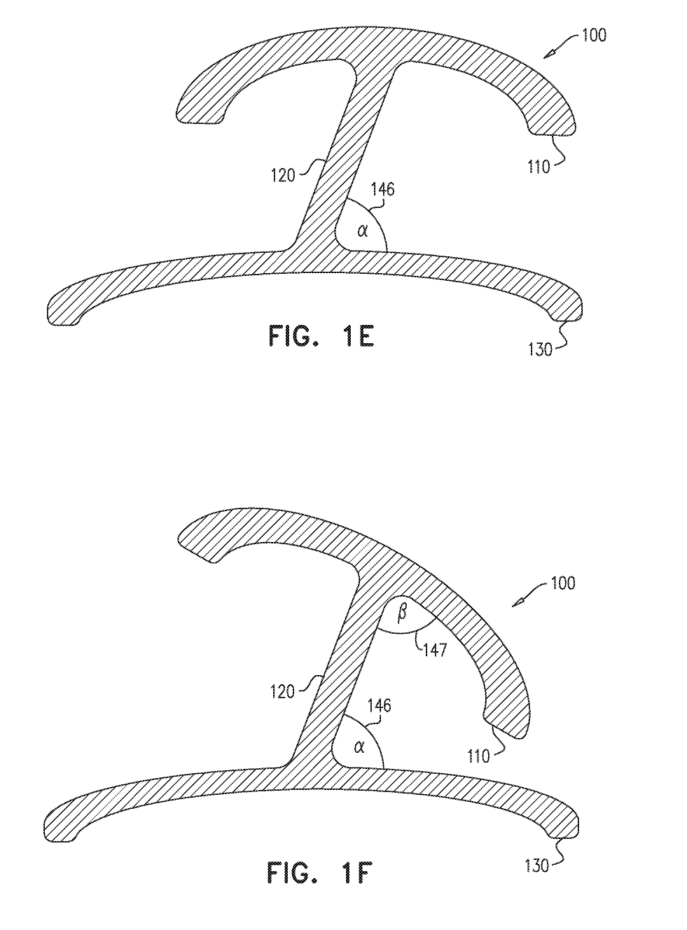

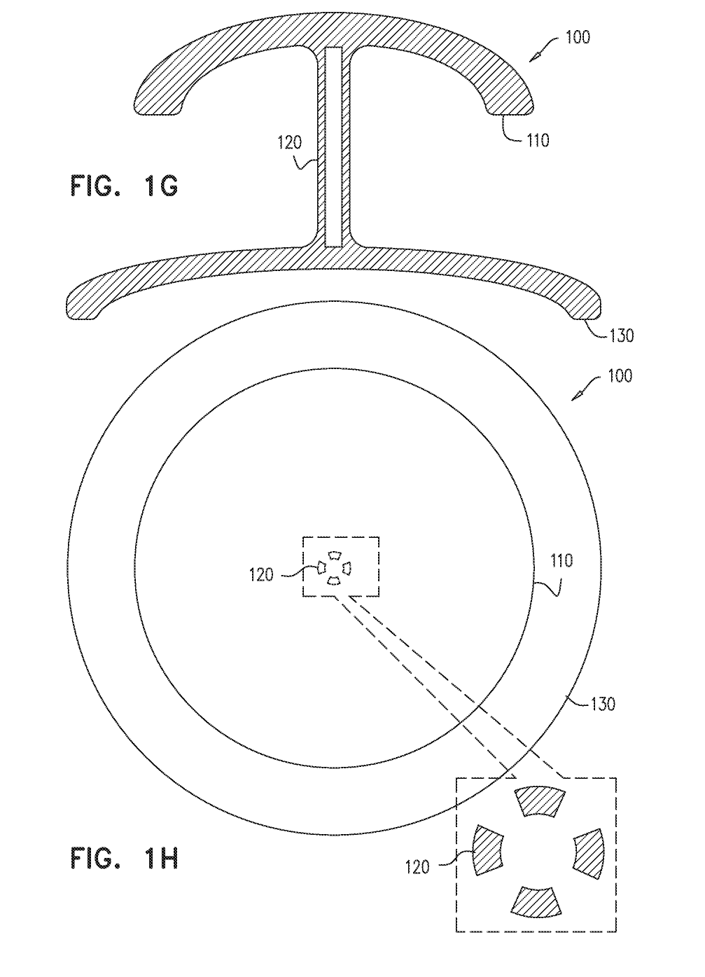

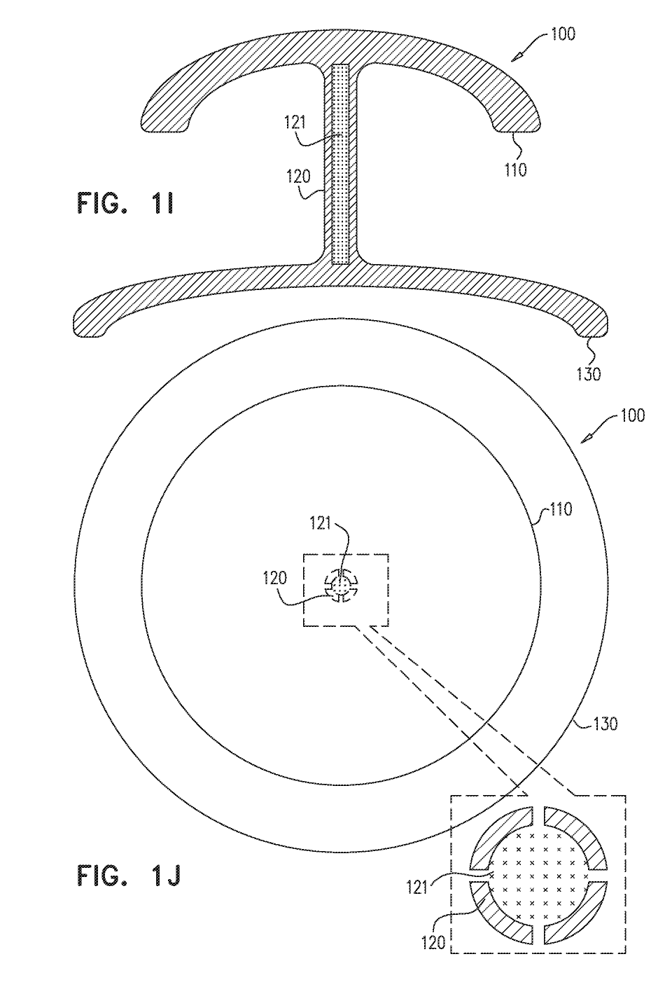

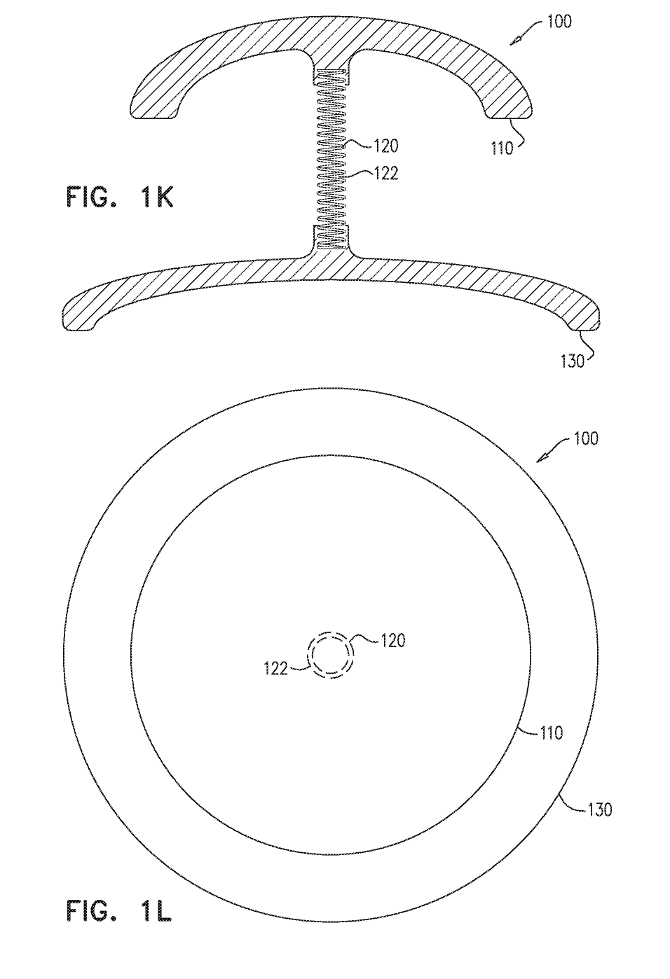

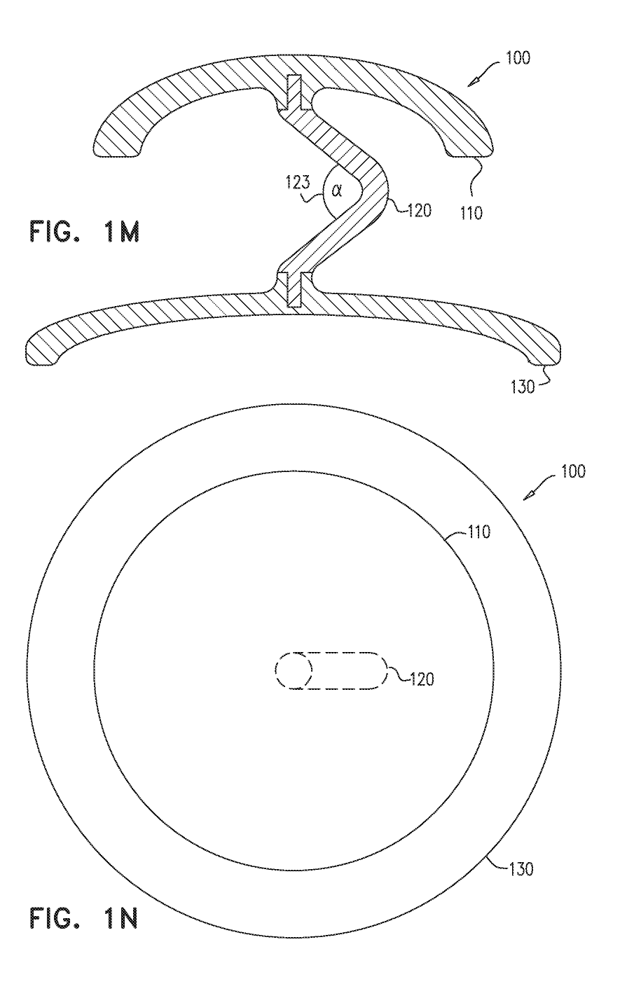

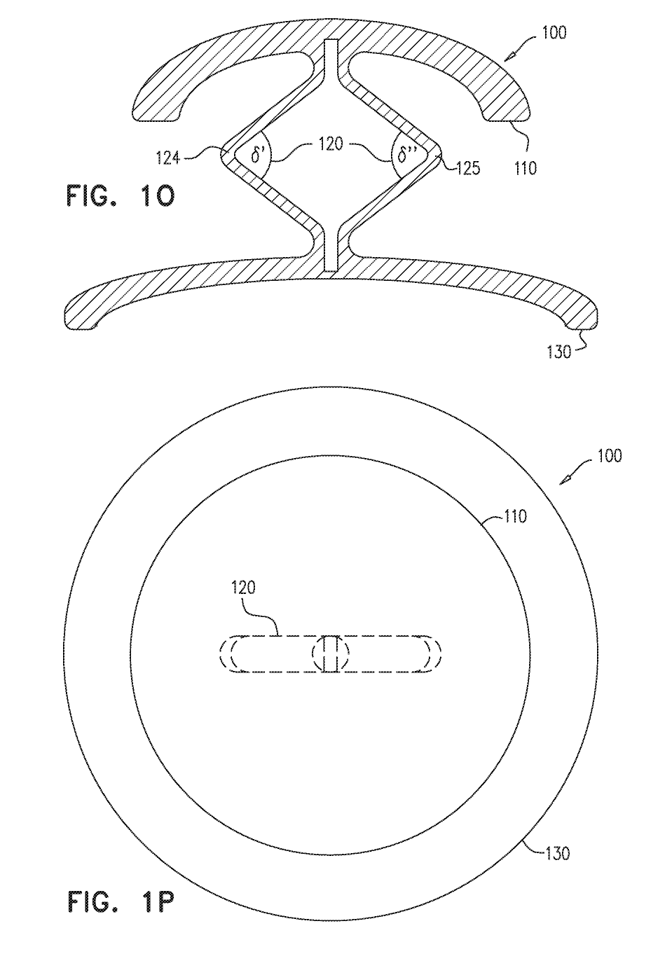

Reference is now made to FIG. 1A and FIG. 1B, which illustrate, respectively, a sectional side view and a pictorial top view of a projecting breast implant 100 according to some embodiments of the present invention. The projecting breast implant shown in FIGS. 1A-1B includes three functional parts: a top breast supporting dome 110, configured to be positioned within a subject's breast parenchyma underneath the nipple-areola complex when implanted, and to hold surrounding breast tissue and overlying skin in a new projecting configuration; a base 130, configured to be in contact with the chest wall of the patient or with tissue adjacent to the chest wall and provide support; and an elongated projecting structure 120 connecting dome 110 and base 130. As shown in FIG. 1A, elongated projecting structure 120 is configured to define how far the breast extends forward from the chest wall, namely to define a new distance between the chest wall and the breast tissue supported and projected outwards by the implant.

In the illustrated embodiment, projecting structure 120 is in the form of a pillar connected on one side thereof to a center of projecting dome 110, and in an opposing side thereof to a center of base 130. In the illustrated embodiment, the diameter of dome 110 is smaller than the diameter of base 130. According to other embodiments, the diameter of the dome may be the same as the diameter of the base.

The width of the projecting structure is smaller than the diameters of the dome and the base. In some embodiments, the width of the projecting structure may range from about 5 mm to about 20 mm. In some embodiments, the diameter of the dome may range from about 20 mm to about 70 mm. In some embodiments, the diameter of the base may range from about 40 mm to about 120 mm.

In some embodiments, thickness of each of the dome and base may range from about 2 mm to about 7 mm. In some embodiments, the dome, the base or both are of a uniform thickness. In other embodiments, the dome, the base or both are of varying thickness. In some embodiments, the dome and the base are of the same thickness. In other embodiments, the dome and the base are of different thicknesses.

In some embodiments, the length of projecting structure may range from about 40 mm to about 150 mm.

FIG. 1B illustrates a pictorial top view of the projecting breast implant of FIG. 1A, showing that projecting dome 110 has a smaller diameter than base 130. According to some embodiments, projecting dome 110 may be asymmetrical. Projecting dome 110 is typically convex, but according to some embodiments it may be flat. According to some embodiments, projecting dome 110 or base 130 may be made of finger like projections rather than a full dome. This is only an example of one of the options related to the relative sizes and shapes of projecting dome 110 and base 130. Various shapes and sizes may be used for projecting dome 110 and base 130 as desired and defined by a manufacturer and for different sizes and types of breast to be implanted with this projecting breast implant.

Reference is now made to FIG. 1C and FIG. 1D, which illustrate sectional side views of some embodiments of projecting breast implant 100 where any of the three parts that constitute the projecting breast implant, namely, dome 110, projecting structure 120 and base 130, may be hollow or filled with a material different from the material forming the outer wall of the part. According to some embodiments, dome 110, projecting structure 120 and base 130 may be integrally formed. According to other embodiments, dome 110, projecting structure 120 and base 130 may be formed as separated parts that are configured to be assembled.

According to some embodiments, dome 110, projecting structure 120 and base 130 may be made of the same material. According to other embodiments, dome 110, projecting structure 120 and base 130 may be made of different materials. According to additional embodiments, each of dome 110, projecting structure 120 and base 130 may be made of a combination of materials. Examples of materials suitable for the breast implant of the present invention include various silicone polymers, polyurethane and others known in the art. These different constructions/compositions can be used, for example, to give one of the implant's components different mechanical properties compared to the other components, to improve overall performance of the breast implant.

In FIG. 1C, for example, projecting structure 120 has a core 140 (body) that is made of, or filled with, a material that is different from the material forming the outer wall (surface) of projecting structure 120. In some embodiments, core 140 of projecting structure 120 may be filled with gas, e.g., air, or with closed-cell silicone foam or other foamy materials to reduce the weight of the breast implant. In other embodiments, core 140 of projecting structure 120 may be made of a substance that has a greater stiffness than the stiffness of the substance forming the outer wall of projecting structure 120. Greater stiffness of core 140 may be advantageous in cases where stronger support is needed, for example with a relatively large and heavy breast. For example, core 140 may be made of silicone of higher stiffness, like a silicone having a higher Shore A Durometer, or another material as defined by the manufacturer.

In FIG. 1D, each of dome 110, projecting structure 120 and base 130 has a core (150, 140 and 145, respectively) made of, or filled with, a material that is different from the material forming the outer wall of each part. In the embodiment illustrated in FIG. 1D, each of core 150, core 140 and core 145 is made of, or filled with, a different material. In some embodiments, core 150 of dome 110 may be filled with gas, e.g., air, or made from a material that is softer than the outer wall of dome 110. Such filling may be used to render projecting dome lighter and/or softer, due to the damping effect of a softer filling material. In some embodiments, core 145 of base 130 may be filled with gas, e.g., air, or made from a material that is softer then the outer wall of base 130, so base 130 can be lighter or can be softer due to the damping effect of a softer filling material 145.

Reference is now made to FIG. 1E and FIG. 1F, which illustrate sectional side views of additional embodiments of projecting breast implant 100, where the implant is designed such that its shape is adjustable and changeable in response to pressure applied to it by surrounding breast tissue upon implantation, according to the size, shape, projection, weight and volume of the operated breast.

In FIG. 1E, acute angle alpha (146) between base 130 and projecting structure 120 is set during the manufacturing process. Acute angle alpha typically ranges from about 60 degrees to about 85 degrees. Following insertion of breast implant 100 into a subject's breast, weight of surrounding breast parenchyma may push projecting dome 110 and projecting structure 120 such that acute angle alpha is changed into a less acute angle, until an equilibrium is reached between the pressure applied by projecting breast implant 100 and the counter-pressure applied by the operated breast. For example, acute angle alpha of 70 degrees (set during the manufacturing process) may change to about 85 degrees following insertion and application of pressure by surrounding tissue.

FIG. 1F shows another embodiment of an adjustable projecting breast implant 100. In the illustrated embodiment, an acute angle beta (147) is defined between dome 110 and projecting structure 120, as well as acute angle alpha (146) between base 130 and projecting structure 120. Both angles are set during the manufacturing process. In some embodiments, acute angle alpha and acute angle beta are the same. In other embodiments, acute angle alpha and acute angle beta are different. Acute angle beta typically ranges from about 60 degrees to about 85 degrees. This embodiment is designed to accommodate for a heavy breast once projecting breast implant is placed within breast parenchyma. In this embodiment there are two degrees of freedom to adopt to breast weight where the weight of breast tissue closer to the chest wall will have a higher impact on acute angle alpha, and the weight of breast tissue more distant from the chest wall will have a higher impact on acute angle beta. Upon implantation, acute angle alpha, acute angle beta or both change in response to the pressure applied to projecting breast implant 100 by surrounding tissue until an equilibrium is reached between the pressure applied to projecting breast implant 100 and the counter-pressure applied by projecting breast implant 100. The result is a desired new projecting position of the breast post operatively.

Reference is now made to FIG. 1G and FIG. 111, which respectively illustrate a sectional side view and a top pictorial view of yet another embodiment of projecting breast implant 100, in which projecting structure 120 is constructed of a plurality (at least two, e.g., two, three, four of more) of sub-structures constructing together a projecting structure according to mechanical properties defined by a manufacturer. In the illustrated embodiment, projecting structure 120 is composed of a plurality of spaced elongated elements. The elongated elements are circularly-arranged, each extending between dome 110 and base 130, such that a cylindrical space is defined in-between.

Reference is now made to FIG. 1I and FIG. 1J, which respectively illustrate a sectional side view and a top pictorial view of the projecting breast implant of FIGS. 1G-111, further showing scar tissue 121 generated within the cylindrical space between the sub-structures of projecting structure 120. Once projecting implant 100 is placed inside an operated breast, the space between the sub-structures constructing projecting structure 120 is filled with breast parenchyma, and scar tissue 121 is generated during postsurgical healing processes. The combination of elastic and plastic properties of the post-operative scar tissue, and the mechanical properties of projecting breast implant 100, defines a composite implant—breast tissue complex, characterized by properties that differ from the properties of each of the entities separately.

Reference is now made to FIG. 1K and FIG. 1L, which respectively illustrate a sectional side view and a top pictorial view of another embodiment of projecting breast implant 100. In the illustrated embodiment, projecting structure 120 is constructed from at least one spring 122 connecting between dome 110 and base 130. Different spring mechanical properties, e.g., a harder or a softer spring, may define different resistance to breast weight once implanted in breast parenchyma, different response to pressure applied to the breast, and different response to changes in body posture and body movement such as during walking and running. The suitable implant will be selected by a surgeon according to need and patient's characteristics. Breast tissue and post-operative scar tissue filling the gaps in the spring structure will define a composite implant-breast tissue complex, characterized by new mechanical properties.

Reference is now made to FIG. 1M and FIG. 1N, which respectively illustrate a sectional side view and a top pictorial view of another embodiment of projecting breast implant 100. In the illustrated embodiment, projecting structure 120 is made of a resilient material manufactured in a bent structure at angle alpha (123), acting as a spring to hold a projected breast in a projecting position after implantation of projecting breast implant 100 in breast parenchyma. Angle alpha typically ranges from about 30 degrees to about 60 degrees. Following implantation, the presence of breast parenchyma filling the triangular space defined by acute angle alpha defines new combined properties to the combination of projecting breast implant 100 and breast parenchyma as a composite implant. The spring like properties of projecting structure 120 allows dynamic movement of the breast reflecting the action-reaction mechanism between the breast and projecting structure 120.

Reference is now made to FIG. 1O and FIG. 1P, which respectively illustrate a sectional side view and a top pictorial view of another embodiment of projecting breast implant 100. In the illustrated embodiment, projecting structure 120 is made of at least two resilient elongated members 124 and 125, each manufactured in a bent structure (acute angles delta′ and delta″, respectively). Elongated members 124, 125 act as springs to hold a projected breast in a projecting position after implantation of projecting breast implant 100 in breast parenchyma. Following implantation, the presence of breast parenchyma filling the triangular spaces defined by acute angles delta′ and delta″ defines new combined properties to the combination of projecting breast implant 100 and breast parenchyma as a composite implant. According to some embodiments, angles delta′ and delta″ may be identical. According to other embodiments, angles delta′ and delta″ may be different. Angles delta′ and delta″ typically range from about 30 degrees to about 60 degrees. The spring like properties of projecting structure 120 allows dynamic movement of the breast reflecting the action-reaction mechanism between the breast and projecting structure 120.

Reference is now made to FIG. 1Q and FIG. 1R, which respectively illustrate a sectional side view and a pictorial perspective view of yet another embodiment of projecting breast implant 100. In the illustrated embodiment, projecting structure 120 comprises two sub-structures, namely a resilient angled structure 150 bent at an angle alpha (153) and a spring structure 155. This is an example for a projecting structure that is made of different sub-structures rather than similar or identical sub-structures, as presented elsewhere in the description. In the illustrated embodiment, the two sub-structures 150 and 155 are each connected to base 130 and dome 110. In other embodiments, a first sub-structure may be connected to both base 130 and dome 110, and a second sub-structure may be connected to either dome 110 or base 130 on one end thereof, while an opposing end thereof is connected to the first sub-structure or remains free. Sub-structures 150 and 155 are configured to support dome 110 in different directions and allow the dome to withstand pressure from different directions simultaneously. Angled structure 150 may be made of a biocompatible resilient material like silicone that allows it to gradually bend such that angle alpha becomes more acute when pressure is applied to it, and gradually return to its original shape as pressure is reduced. Spring structure 155 is made of a biocompatible resilient material like silicone that allows it to gradually bend when pressure is applied to it and gradually return to original shape as pressure is reduced. In a spring like substructure, such as spring structure 155, pressure is absorbed maximally when applied at a right angle to a center of dome 110. Angled structure 150 may have various alpha angles as designed by a manufacturer to have different resiliency to pressure applied to it. According to some embodiments, angled structure 155 may be made of more than one polymer where each polymer has different mechanical properties giving angled structure 155 mechanical properties combining the different properties of each polymer. Angled structure 155 is designed to absorb pressure applied to dome 110 not only at a right angle but also laterally to spring structure 155.

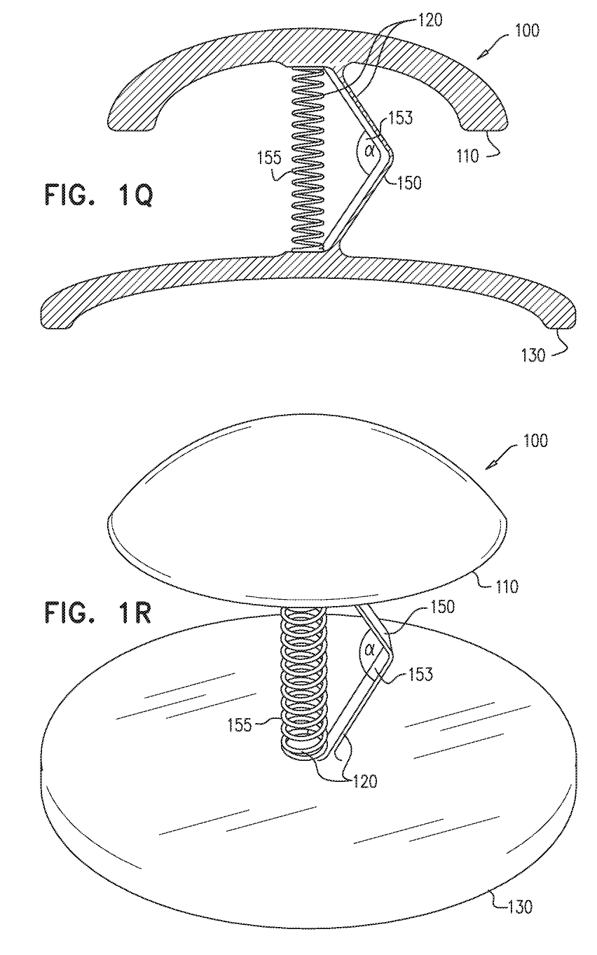

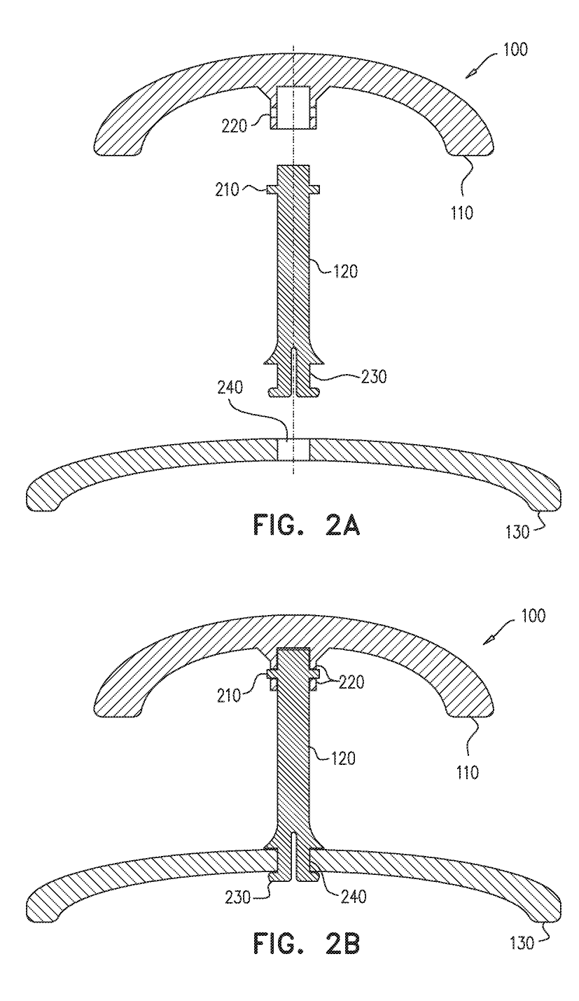

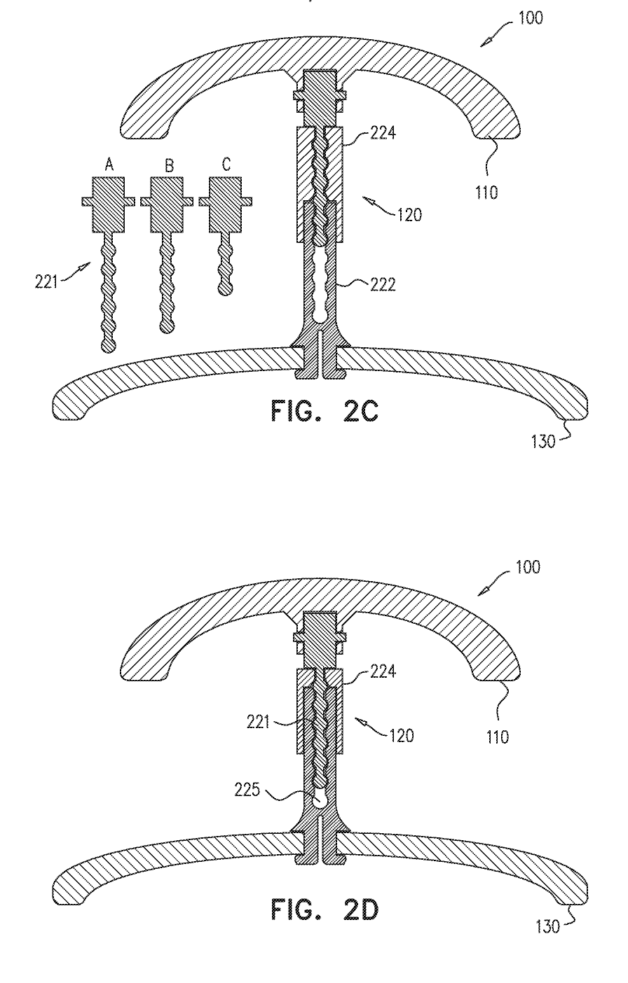

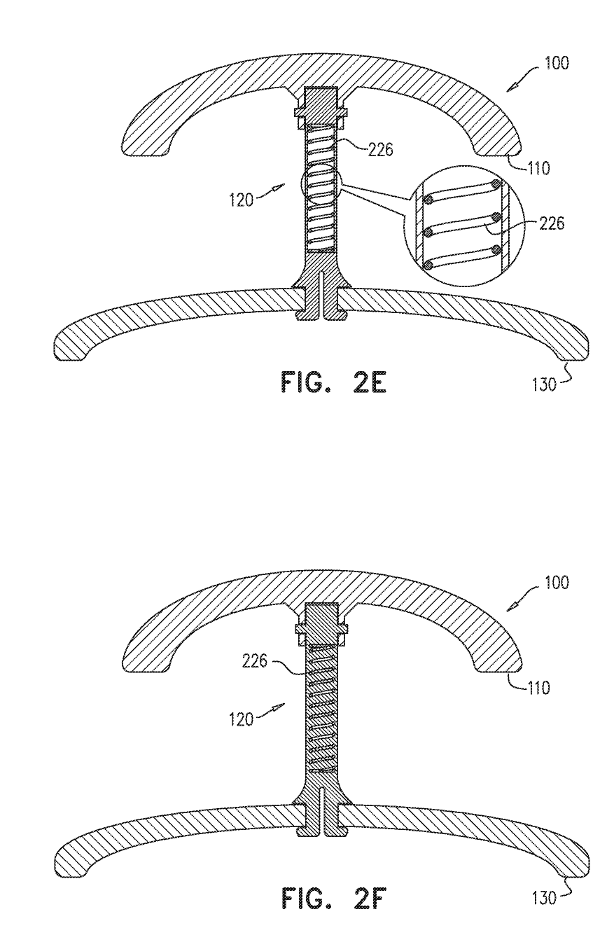

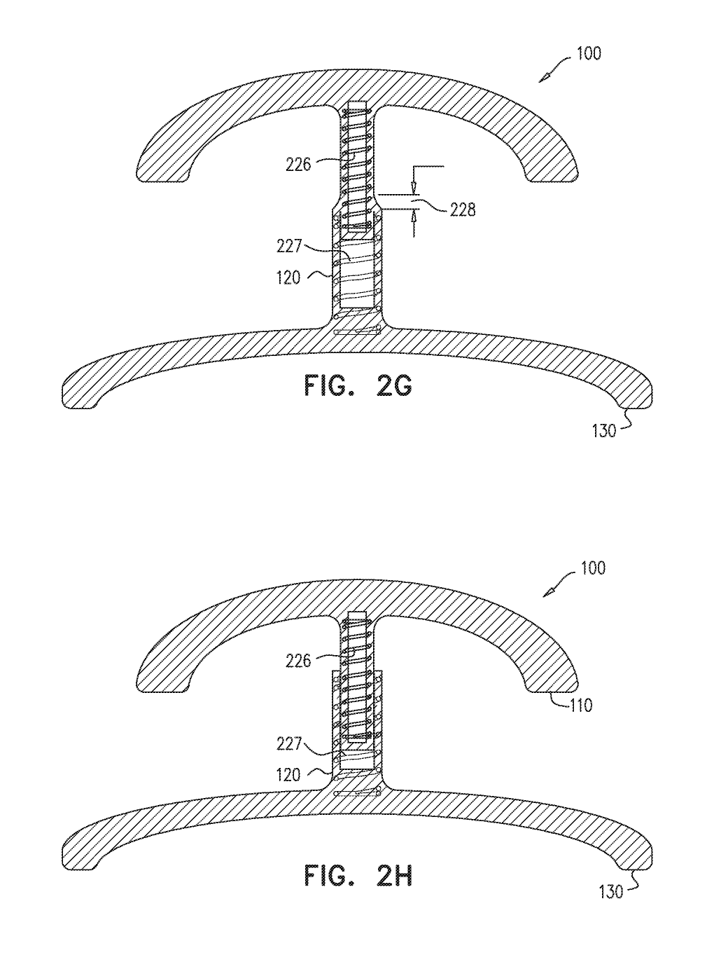

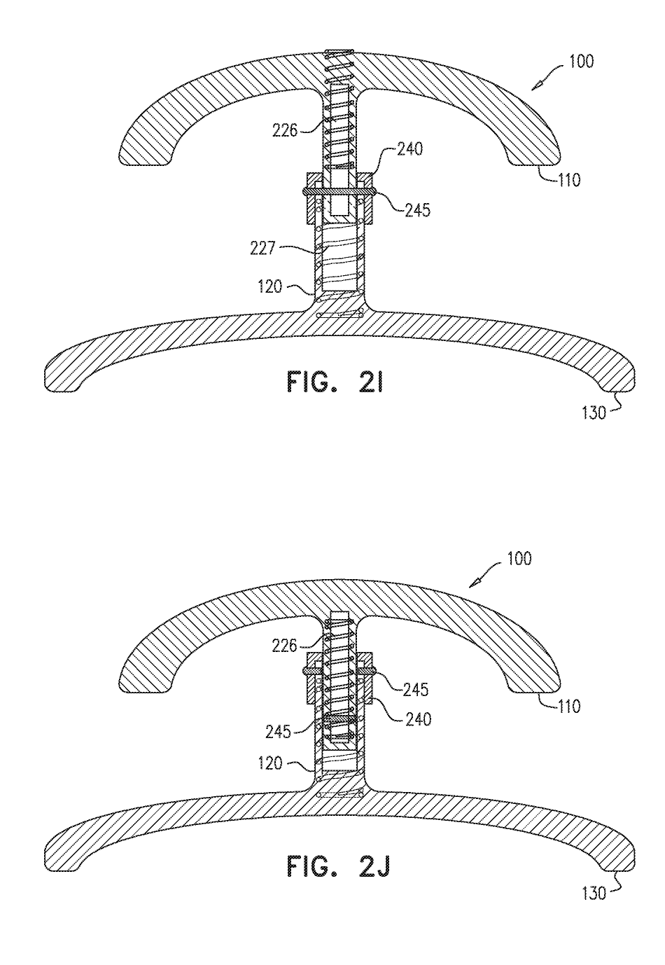

Reference is now made to FIG. 2A and FIG. 2B, which illustrate sectional side views of a projecting breast implant 100 according to additional embodiments of the present invention. In the illustrated embodiment, the three parts that constitute projecting breast implant 100, namely, dome 110, projecting structure 120 and base 130, are manufactured as separate parts configured to assemble to construct the complete projecting breast implant 100. In the illustrated embodiment, projecting structure 120 is in the form of a pillar comprising a distal end configured to connect to base 130 and a proximal end configured to connect to dome 110.

FIG. 2A shows the three components separately. Dome 110 and the proximal end of projecting pillar 120 are configured to assemble via a dome-pillar locking mechanism, comprising a dome portion 220 and a pillar portion 210. Base 130 and the distal end of projecting structure 120 are configured to assemble via a pillar-base locking mechanism, comprising a pillar portion 230 and a base portion 240.

In FIG. 2B the three components of projecting breast implant 100 are shown when combined and locked in place as a functional projecting breast implant. The locking mechanisms displayed here are merely exemplary locking mechanisms between parts and any other suitable locking mechanism may be used.

Reference is now made to FIG. 2C and FIG. 2D, which illustrate sectional side views of another embodiment of projecting breast implant 100, wherein projecting structure 120 comprises a fixed element 222 and a replaceable element 221 that are configured to adjustably connect such that the length of projecting structure 120 is varied. Thus, the projection of the implant may be adjusted by a surgeon during operation.

In FIG. 2C projecting structure 120 includes a fixed element 222 in the form of a socket connected to base 130, and a replaceable element 221. Replaceable element 221 comprises an upper end configured to connect to dome 110 and a lower end configured to fit within fixed element 222. Fixed element 222 and replaceable element 221 are shown in FIG. 2C in association with one another, where replaceable element 221 is partially accommodated in fixed element 222.

During operation, a series of replaceable elements 221A, 221B, and 221C of variable lengths may be provided. A surgeon may select a suitable replaceable element from among the series of replaceable elements, and connect it to dome 110 via the upper end of the replaceable element. The lower end may be fit within fixed element 222 and moved along fixed element 222 until the desired length of projecting structure 120 is obtained.

The design of projecting structure 120 may be changed such that the fixed element is the upper portion configured to connect to the projecting dome, and the replaceable element is the lower portion configured to connect to the supporting base.

In FIG. 2D projecting breast implant 100 is shown in an assembled configuration, ready for implantation in a subject's breast. Replaceable element 221 is placed inside fixed element 222. Replaceable element 221 may be partially or fully accommodated in fixed element 222, as needed to obtain a desired projection. In the illustrated embodiment, replaceable element 221 is partially accommodated in the socket such that a gap 225 is left at the bottom of the socket. Gap 225 indicates that a higher projection of projecting breast implant 100 was needed by the operating surgeon to get the desired breast projection, dictated inter alia by breast size, degree of ptosis and the patient's wish. Projecting implant 100 shown in FIG. 2D further comprises a sliding sleeve 224 configured to secure and lock replaceable element 221 in place and prevent it from sliding after its positioning within fixed element 222 to obtain a desired projection. In some embodiments, the sliding sleeve secures and locks the replaceable element by applying external pressure on the fixed element. Other means enabling changing the length of the projecting structure, and accordingly the projection of the projecting breast implant, may be used. Other designs may be included, for example springs, hydraulic pumps, pneumatic mechanisms and other mechanical designs known in the art.

Reference is now made to FIG. 2E and FIG. 2F, illustrating sectional side views of other embodiments of projecting breast implant 100. In the illustrated embodiments, projecting breast implant 100 is composed of separate dome 110, base 130 and projecting structure 120 that are configured to assemble to construct projecting breast implant 100, where projecting structure 120 includes a spring 226 configured to allow compression of projecting structure 120. Spring 226 may be manufactured from a polymer material, e.g. silicone, or others known in the manufacturing of long term implantable devices.

In FIG. 2E projecting structure 120 comprises an internal lumen, and spring 226 is placed inside the internal lumen thereby providing support and defining new mechanical properties of projecting structure 120. In FIG. 2F spring 226 is embedded within projecting structure 120. Any other combinations and variations may be used to place a spring in the projecting structure, for example incorporating the spring into the wall of the projecting structure, or any other design.

Reference is now made to FIG. 2G and FIG. 2H, illustrating sectional side views of another embodiment of projecting breast implant 100 where projecting structure 120 is telescopic and is configured to transiently change a distance between dome 110 and base 130 in response to pressure applied to the breast. In the illustrated embodiment, projecting structure 120 includes two springs of different diameters, namely first spring 226 and second spring 227, where the diameter of first spring 226 is smaller than the diameter of second spring 227. First spring 226 is connected on one end thereof to dome 110 and the other end thereof is partially located within second spring 227. Second spring 227 is connected on one end thereof to base 130 and the other end accommodates a portion of first spring 226. Projecting structure 120 further includes a breaking area 228 at the interface between the first and second springs that is configured to be responsive to pressure applied to the breast. When the pressure exceeds a certain threshold the breaking area allows collapse of projecting structure 120, via sliding of first spring 226 into second spring 227. In the collapsed state (FIG. 2H), a distance between dome 110 and base 130 is smaller than the distance at the extended state. When the pressure is reduced or removed, first spring 226 slides outside of second spring 227 to restore a full length projecting structure 120.

Reference is now made to FIG. 2I and FIG. 2J, illustrating sectional side views of an implant as shown in FIGS. 2G-H, that further comprises a pin 245 and a bridging area 240. Bridging area 240 and pin 245 hold projecting structure 120 in a full extended projection. Upon pressure applied to it that is larger than a threshold pressure pin 240 can hold, it breaks and first spring 226 slides into second spring 227. Pin 245 then locks first and second springs in the new configuration.

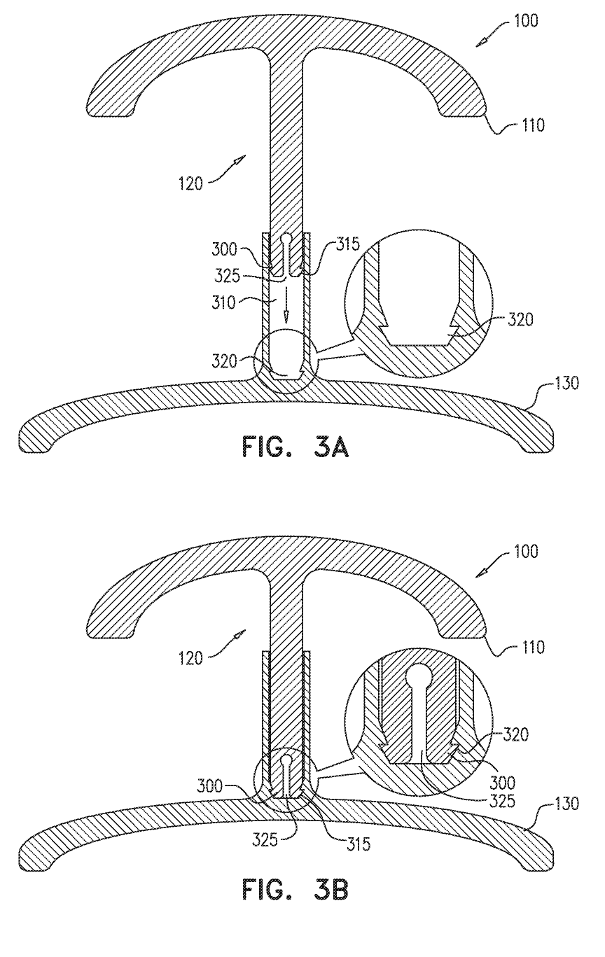

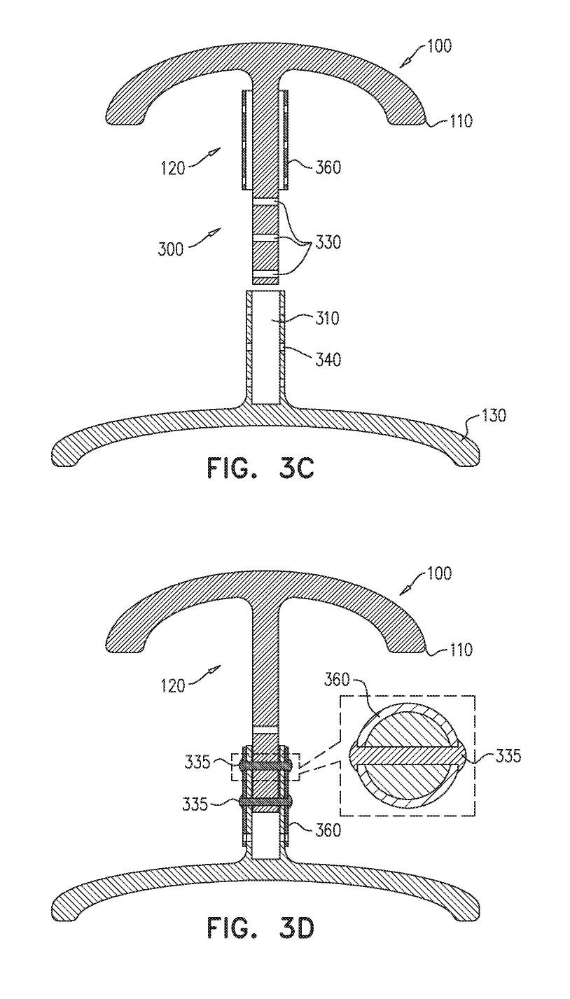

Reference is now made to FIG. 3A and FIG. 3B, illustrating sectional side views of an additional embodiment of projecting breast implant 100, comprising integrally formed dome 110 and projecting structure 120 configured to assemble to base 130 via a snap-fit locking mechanism.

In the illustrated embodiment, base 130 comprises a cylindrical hollow holding member 300 extending upwardly from the center of base 130, configured to accommodate the distal end of projecting structure 120. Holding member 300 comprises an internal snap-in recess 320, configured to receive and secure a matching protruding edge 315 on the distal end of projecting structure 120. Projecting structure 120 comprises an opening 325 at its distal end configured to enable the distal end to resiliently deflect inwardly (i.e., towards the opening) upon engagement with snap-in recess 320, until protruding edge 315 is secured within snap-in recess 320.

Reference is now made to FIG. 3C and FIG. 3D, illustrating sectional side views of an additional embodiment of projecting breast implant 100 where a different locking mechanism is used in the assembly between dome 110 and projecting structure 120, manufactured as a single element, and base 130. FIG. 3C shows the element composed of dome and projecting structure disassembled from the base. Projecting structure 120 comprises a plurality of circumferential apertures 330 at a distal end thereof. The distal end of projecting structure 120 is configured to engage within a cylindrical hollow accepting member 310 extending upwardly from the center of base 130. Accepting member 310 similarly comprises a plurality of circumferential apertures 340, matching circumferential apertures 330 of projecting structure 120 (at identical distances) such that upon engagement of projecting structure 120 and accepting member 310, circumferential apertures 330 are aligned with circumferential apertures 340.

In FIG. 3D dome 110 and projecting structure 120 are assembled with base 130, and locked in place via locking pins 335 and compressing sleeve 360. Locking pins 335 pass through apertures in the walls of compressing sleeve 360, through circumferential apertures 340 in the wall of accepting member 340 and through circumferential apertures 330 in the distal end of projecting structure 120. Insertion of locking pins 335 through the apertures of compressive sleeve 360 is advantageous in overcoming banding and to ease the sliding of locking pins 335 into position. This assembly and locking mechanism allows a surgeon to change the degree of projection of the projecting breast implant according to need during surgery. For example, in the illustrated embodiment, each of projecting structure 120 and accepting member 310 comprises three circumferential apertures, and when assembled, only the top two pairs of apertures are engaged and locked, resulting in a higher projection compared to a case where all three pairs of apertures are engaged and locked.

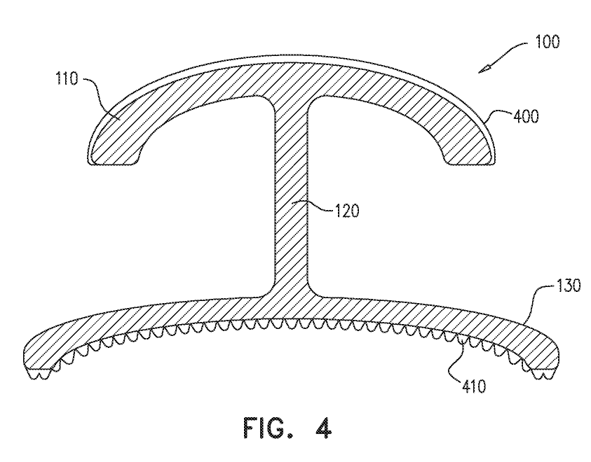

Reference is now made to FIG. 4, illustrating a sectional side view of a projecting breast implant in accordance with some embodiments of the present invention, which comprises outer surface covering layers. In the illustrated embodiment, the projecting breast implant comprises a covering layer on top of the dome, and a base layer underneath the support base. In the illustrated embodiment dome 110, projecting structure 120 and base 130 are made from a first material, and the uppermost surface of dome 110 is covered with a top layer 400 made of a second material that is different from the first material. Top layer 400 is configured to change the tactility of projecting breast implant 100 through the subcutaneous tissues and skin covering it. The material forming top layer 400 may be, as an example, a material that is softer than the material forming the dome, projecting structure and base, such as a soft silicone polymer, closed-cell silicone foam, polyurethane or any other material known and approved for long-term implantation. Projecting breast implant 100 illustrated in FIG. 4 further comprises a base layer 410 at the bottom surface of base 130. The material forming base layer 410 may be softer than the material base 130 is made of. Base layer 410 may have a non-smooth, or textured, surface at the bottom to improve the contact and adherence to the chest wall and to reduce sliding of projecting breast implant 100 over the chest wall.

Reference is now made to FIG. 5, illustrating a schematic lateral cross section of a breast 500. Chest wall 510 lies underneath the breast. Breast 500 includes covering skin 530 and breast parenchyma 520. Breast 500 requires correction of sagging.

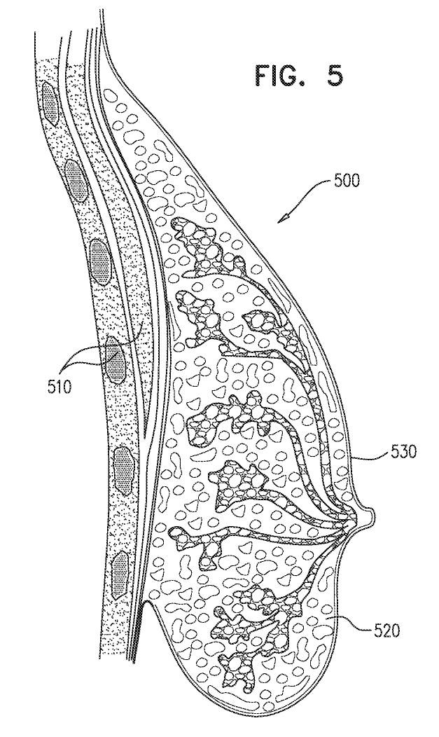

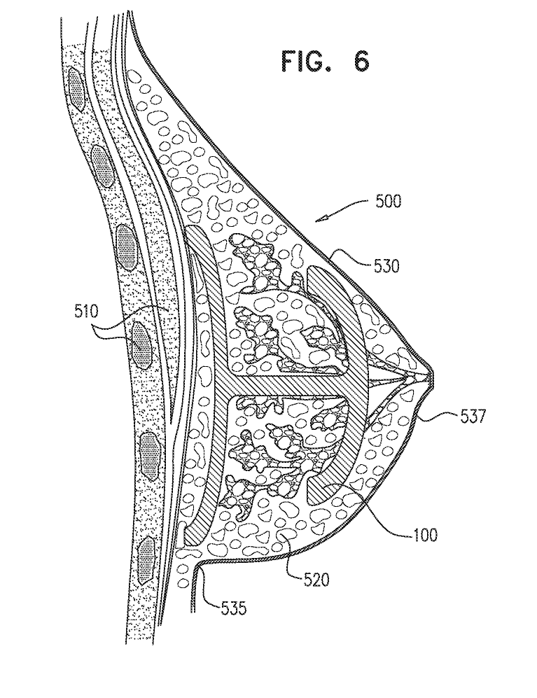

Reference is now made to FIG. 6, illustrating a schematic lateral cross section of breast 500 with a projecting breast implant 100 implanted in it, according to some embodiments. Projecting breast implant 100 lifts breast parenchyma 520 and overlying skin 530 to a desired position and shape. The projecting breast implant may be inserted into the breast through a skin incision placed in the infra-mammary fold 535 where a precise size pocket is developed in the sub-mammary plain; a vertical dissection is carried out through breast parenchyma 520 and a dissection plain is developed under skin and breast parenchyma leaving ample tissue coverage over projecting breast implant 100. The dissection plain developed under skin and breast parenchyma leaving ample tissue coverage over projecting breast implant 100 can be performed through a skin incision at the skin-areola border 537.

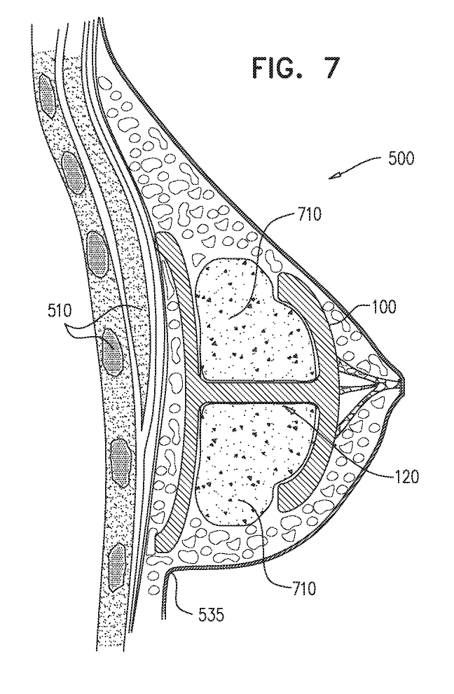

Reference is now made to FIG. 7, illustrating a schematic lateral cross section of breast 500 implanted with a projecting breast implant 100 according to some embodiments combined with a torus, ‘bagel-like’, structure 710, thus providing both projection and augmentation of the breast. Torus structure 710 surrounds projecting structure 120. Torus structure 710 is shown in its filled configuration, where it is filled with a biocompatible material. In the illustrated embodiment, torus structure 710 completely fills a space between the dome and base of projecting breast implant 100. For insertion of the implant into a subject's breast, dissection of a pocket can be carried out through an infra-mammary skin incision 535 at a sub-mammary plain or sub-pectoral plain. The breast implant augments and adds to the projection of the breast as desired.

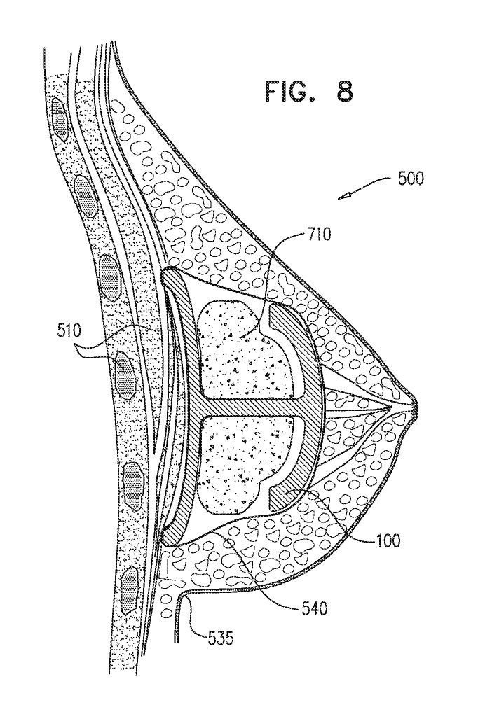

Reference is now made to FIG. 8, illustrating a schematic lateral cross section of breast 500 implanted with a projecting breast implant 100 according to some embodiments combined with a torus, ‘bagel-like’, structure 710, implanted as a replacement breast implant after a previous implant has been removed. Through a sub-mammary skin incision 535 an old breast implant can be removed. A new breast implant combined of projecting breast implant 100 and a torus, ‘bagel-like’ augmenting structure 710 can then be inserted into a breast capsule 540 formed around the old breast implant.

Breast implants according to embodiments of the present invention may include a code identifier (label) on their surface or embedded therein, for non-invasively identifying the implants after they are implanted. According to one embodiment the code identifier may be printed during the manufacturing process. “Ink” materials may include, e.g., polymers, which can be distinguished and detected by optical, electro-optical, electromagnetic, ultrasonic detection means, or other detection means known in the art. For example, a printing material may include a radio-opaque material like barium sulfate, may contain gas bubbles to be detected by ultrasound, may contain a color different from the color of the implant layer on which it is printer or in which it is embedded to be detected optically, may contain magnetic material defining a different magnetic imprint for each code, or any combination of the above, but not limited to the above. According to another embodiment, the code may be cut by laser or any mechanical cutting device or method from a sheath made of the above described “ink” materials. The code may be generated by a computer, either randomly or according to a pre-determined algorithm. The code may be a printable character or any drawing or shape. The code may be saved to a computerized database to be retrieved when needed through a local network or over the web. The code can advantageously be retrieved non-invasively from the implant while the implant is still implanted in the patient, without the need to extract the implant by surgery. Once the code is retrieved some or all information regarding the implant, the patient and the operating physicians can be retrieved over the web or in any other method as allowed by authorities.

What we claim is:

1. A breast implant comprising:
a base having a first diameter, said base is configured to rest against a subject's chest wall when implanted;
a dome having a second diameter, said dome is configured to be positioned within breast parenchyma underneath nipple-areola complex when implanted; and
an elongated projecting structure extending between said base and said dome, wherein said implant is configured to be inserted into a subject's breast as an internal supporting skeleton and to affect the projection of breast.

2. The implant of claim 1, wherein said elongated projecting structure comprises a plurality of resilient elongated elements, one or more of said plurality of resilient elongated elements extend between said dome and said base; and/or wherein said elongated projecting structure comprises one or more pillars and/or at least one spring.

3. (canceled)

4. (canceled)

5. The implant of claim 1, wherein said elongated projecting structure comprises differently shaped projecting structures having different mechanical properties.

6. (canceled)

7. (canceled)

8. The implant of claim 1, wherein said first diameter is larger than said second diameter.

9. (canceled)

10. The implant of claim 1, wherein at least one of said base, said elongated projecting structure and said dome is formed as a separate component, which is configured to be assembled with remaining component(s) to form said implant.

11. The implant of claim 1, wherein said implant is at least partially made of or coated with human biocompatible materials.

12. (canceled)

13. The implant of claim 1, wherein said dome comprises one or more radial ribs.

14. (canceled)

15. The implant of claim 1, wherein said implant is variably configured to define projection of the breast during surgery.

16. The implant of claim 1, wherein said implant is configured for altering the projection of the breast post-surgery.

17. The implant of claim 1, wherein an angle between said dome and said elongated projecting structure and/or an angle between said base and said elongated projecting structure can be any angle.

18. (canceled)

19. The implant of claim 1, wherein an angle between said dome and said elongated projecting structure is changeable in response to applying external pressure by surrounding breast tissue.

20. The implant of claim 1, wherein an angle between said base and said elongated projecting structure is changeable in response to applying external pressure by surrounding breast tissue.

21. The implant of claim 1, wherein said dome, said elongated projecting structure and said base are manufactured from materials having different mechanical and/or chemical properties.

22. (canceled)

23. The implant of claim 1, wherein said dome, said base, and said elongated projecting structure comprises a body and an outer surface, said body and said outer surface are made from different materials.

24. The implant in claim 1, wherein said elongated projecting structure is telescopic and is configured to transiently change a distance between said dome and said base in response to pressure applied to the breast.

25. (canceled)

26. (canceled)

27. The implant of claim 1, wherein said dome and said base have different outer surface layer tactile feedback.

28. (canceled)

29. (canceled)

30. (canceled)

31. (canceled)

32. The implant of claim 1, further comprising a torus structure configured to surround said elongated projecting structure for further facilitating breast projection and for providing breast augmentation; wherein said torus structure is filled or fillable with a biocompatible material; wherein said biocompatible material has a specific gravity lower than 0.4 gr/cc.

33. (canceled)

34. (canceled)

35. (canceled)

36. (canceled)

37. (canceled)

38. A breast implant kit comprising:
(i) an internal skeleton comprising:
a base having a first diameter, said base is configured to rest against a subject's chest wall when implanted;
a dome having a second diameter, said dome is configured to be positioned within breast parenchyma underneath nipple-areola complex when implanted; and
an elongated projecting structure extending between said base and said dome; and
(ii) a torus structure configured to surround said elongated projecting structure and at least partially fill a gap between said base and said dome.

39. (canceled)

40. A method of affecting the projection of a breast, the method comprising;
(i) inserting into the breast an internal skeleton comprising:
a base having a first diameter, said base is configured to rest against a subject's chest wall when implanted;
a dome having a second diameter, said dome is configured to be positioned within breast parenchyma underneath nipple-areola complex when implanted; and
an elongated projecting structure extending between said base and said dome; and
(ii) adjusting breast projection.

41. (canceled)

42. (canceled)

43. The method of claim 40, further comprising inserting a torus structure surrounding the projecting structure for further adjusting breast projection and for providing breast augmentation; and filling the torus structure with a filler.

44. (canceled)
